(19) United States
(12) Patent Application Publication
(10) Pub. No.: US 2006/0228416 A1
(43) Pub. Date: Oct. 12, 2006

(54) Methods for modulating topical inflammatory response

(76) Inventors: Marie-Pierre Faure, Ville St. Laurent (CA); Kirill Shingel, Brossard (CA)

Correspondence Address: KIRKPATRICK & LOCKHART NICHOLSON GRAHAM LLP, STATE STREET FINANCIAL CENTER, ONE LINCOLN STREET, BOSTON, MA, 02111-2950, US

(21) Appl. No.: 11/398,919

(22) Filed: Apr. 6, 2006

Related U.S. Application Data
(60) Provisional application No. 60/668,675, filed on Apr. 6, 2005.

(51) Int. Cl. A61K 9/14; A61K 38/16; A61K 38/38

(52) U.S. Cl. 424/486; 514/2

(57) ABSTRACT

A method of modulating topical inflammatory response is disclosed. The method generally includes the selective removal of certain proteins, e.g., one or more cytokines such as interleukin-1β (IL-1β) and/or interleukin-6 (IL-6), from a topical site without substantially altering the local concentrations of other proteins that may be present at or near the topical site. Other proteins that are present at or near the topical site can include serum albumin, fibrinogen, and immunoglobin G (IgG). Hydrogel compositions that can be used to practice the methods of the invention are provided. The invention further provides methods of preparing such hydrogel compositions.

This application claims the benefit of the filing date of U.S. Provisional Patent Application Ser. No. 60/668,675, filed on Apr. 6, 2005, the entire disclosure of which is incorporated by reference herein for all purposes.

### TECHNICAL FIELD

The invention generally relates to methods of reducing the duration and/or intensity of a topical inflammatory response. More specifically, the invention relates to methods of preventing, moderating, and/or shortening topical inflammation by selectively removing certain proteins, e.g., one or more cytokines, from a topical site. The invention further provides hydrogel compositions and methods of making the same that are useful for the practice of the methods of the invention.

### BACKGROUND

The wound healing process involves a complex series of biological interactions at the cellular level and is generally considered to occur in several stages known as the “healing cascade.” During the inflammatory phase, fibroblast cells are stimulated to produce collagen. During the proliferative phase, reepithelialization occurs as keratinocytes migrate from wound edges to cover the wound, and new blood vessels and collagen are laid down in the wound bed. Finally, during the maturation phase, collagen is remodeled into a more organized structure, eventually resulting in the formation of new skin, possibly accompanied by a scar.

It has been suggested that by shortening or completely bypassing the inflammatory phase of the wound healing process, wounds can heal faster and possibly without any scarring. See, e.g., Martin, P. (1997), SCIENCE, 276: 75-81; Hopkinson et al. (1994), J. CELL. SCI., 107(5): 1159-67; Whitby et al. (1991), DEVELOPMENT, 112: 651-68; Adzick et al. (1985), J. PEDIATR. SURG., 20: 315-19. Although a large number of topical and systemic pharmaceuticals have been developed to reduce topical inflammation, most of them only relieve symptoms associated with an inflammatory response. Many of them, such as steroid creams, produce side effects and should be used sparingly.

Thus there is a need to develop methods and compositions that can more effectively modulate topical inflammatory response, and in turn accelerate and/or improve wound healing.

### SUMMARY OF THE INVENTION

It has been discovered that inflammation at a topical site can be modulated by selectively removing certain proteins from the topical site.

According to one aspect of the invention, a method of moderating and/or shortening a topical inflammatory response generally includes removing one or more cytokines from a topical site without substantially altering at the topical site the local concentration of one or more plasma proteins. The one or more cytokines can include at least one of interleukin-1β and interleukin-6. Each of the one or more cytokines can have a molecular weight of less than about 60 kDa. The one or more plasma proteins can include at least one of serum albumin, immunoglobulin G, and fibrinogen. The method can include hydrating the topical site. The method also can include contacting the topical site with a physiological buffer, an antimicrobial, and/or an anticoagulant. The method can include restoring the local osmolarity of the topical site.

In some embodiments, the topical site can be intact skin or an open wound. When the topical site is an open wound, the method can include dissolving an effective amount of fibrinogen at the open wound. The method can accelerate and/or improve the healing of the open wound, for example, by accelerating wound closure, increasing the reepithelialization rate, and/or preventing a scar at the topical site.

In some embodiments, the method can be carried out by applying a hydrogel composition to the topical site. The hydrogel composition typically can include a protein component and a biocompatible polymer component, wherein the protein component is covalently crosslinked by the biocompatible polymer component. The protein component can include one or more proteins such as bovine serum albumin, human serum albumin, lactalbumin, ovalbumin, soy albumin, pea albumin, hydrolyzed soy protein, hydrolyzed wheat protein, casein, and combinations thereof. The biocompatible polymer component can include polyethylene glycol (PEG) or a derivative of PEG.

In certain embodiments, the hydrogel composition can be hydrated when applied to the topical site. For example, at least 90% by weight of the hydrogel composition can be water. The hydrogel composition can include an antimicrobial and/or an anticoagulant. For example, the hydrogel composition can include an anticoagulant such as ethylenediaminetetracetic acid (EDTA) or a salt thereof, and/or an antimicrobial such as diazolidinyl urea and iodopropynyl butylcarbamate.

In some embodiments, the hydrogel composition can include a polymeric backing. The polymeric backing can be attached to the hydrogel composition without an adhesive. The hydrogel composition can be non-adherent to the topical site. The surface of the hydrogel composition can be resistant to adsorption of serum albumin, immunoglobulin G, and/or fibrinogen.

Another aspect of the invention relates to a method of preparing a hydrogel composition suitable for use in the method of moderating and/or shortening a topical inflammatory response described above. The method generally includes reacting a protein component with a bifunctional biocompatible polymer. The protein component can be any of the proteins mentioned above. The bifunctional biocompatible polymer can be a bifunctional polyethylene glycol, such as a dinitrophenylcarbonyl polyethylene glycol, a dichlorosulfonyl polyethylene glycol, a dichloroacetylsulfonyl polyethylene glycol, a dichlorosulfonyl ethylsulfonyl polyethylene glycol, a diphenylcarbonyl polyethylene glycol, a ditoluenesulfonyl polyethylene glycol, a disuccinimidyl polyethylene glycol, a dimaleimidyl polyethylene glycol, a diisocyanato-polyethylene glycol, or a divinylsulfonamido-polyethylene glycol. The method also can include converting a biocompatible polymer into a bifunctional biocompatible polymer before the reacting step. The converting step can be conducted in a solvent or in a solvent-free environment.

The invention also relates to a method of treating a topical inflammatory response by applying a hydrogel composition including a protein component and a biocompatible polymer component, where the protein component is covalently crosslinked by the biocompatible polymer component; and removing biological molecules having a molecular weight of less than about 60 kDa.

The foregoing, and other features and advantages of the invention as well as the invention itself, will be more fully understood from the following figures, description, and claims.

### BRIEF DESCRIPTION OF FIGURES

This patent or application contains at least one drawing executed in color. Copies of this patent or patent application publication with color drawing(s) will be provided by the United States Patent and Trademark Office upon request and payment of the necessary fee.

A skilled artisan will understand that the drawings described below are for illustration purposes only and are not intended to limit the scope of the present teachings in any way.

FIGS. 1a-f are color photographs of a partial thickness wounds treated with an embodiment of a hydrogel composition of the invention. FIG. 1 shows the appearance of the wound in color on day 0 (a), day 2 (b), day 4 (c), day 6 (d), day 9 (e), and day 11 (f), respectively.

FIGS. 2a-f are color photographs of another partial thickness wounds treated with an embodiment of a hydrogel composition of the invention. FIG. 2 shows the appearance of the wound in color on day 0 (a), day 2 (b), day 4 (c), day 6 (d), day 9 (e), and day 11 (f), respectively.

FIGS. 3a-d are color photographs of yet another partial thickness wound treated with an embodiment of a hydrogel composition of the invention. FIG. 3 shows the appearance of the wound in color on day 0 (a), day 2 (b), day 4 (c), and day 6 (d), respectively.

FIGS. 4a-d are color photographs of a partial thickness wound treated with a comparative dressing. FIG. 4 shows the appearance of the wound in color on day 0 (a), day 2 (b), day 4 (c), and day 6 (d), respectively.

FIGS. 5a-e are color photographs of a partial thickness wound treated with another comparative dressing. FIG. 5 shows the appearance of the wound in color on day 0 (a), day 2 (b), day 4 (c), day 6 (d), and day 7 (e), respectively, after scabs have been removed from the wound bed.

FIGS. 6a-e are color photographs of a partial thickness wound treated with yet another comparative dressing. FIG. 6 shows the appearance of the wound in color on day 0 (a), day 2 (b), day 4 (c), day 6 (d), and day 9 (e), respectively, after scabs have been removed from the wound bed.

FIG. 7 is a graphical representation of the rate of wound closure as observed in partial-thickness wounds treated with an embodiment of a hydrogel composition of the invention and three other comparative dressings.

FIG. 8 is another graphical representation of the rate of reepithelialization as observed in partial-thickness wounds treated with an embodiment of a hydrogel composition of the invention and three other comparative dressings.

FIG. 9 is a graphical representation of changes in wound size over time for full-thickness wounds treated with an embodiment of a hydrogel composition of the invention and three other comparative dressings.

FIGS. 10a-d are color histological photographs of partial-thickness wounds treated with an embodiment of a hydrogel composition of the invention and three other comparative dressings after complete re-epithelialization (i.e., on days 6-7 depending on the dressing used).

FIGS. 11a-b are color histological photographs of full-thickness wounds on day 10 after treatment with (a) an embodiment of a hydrogel composition of the invention and (b) dry gauze.

FIGS. 12a-c are graphical representations of the amount of proteins extracted from an embodiment of a hydrogel composition of the invention and two other comparative dressings after 24 hours of application to a partial thickness wound over the course of healing.

FIG. 13 is an HPLC chromatogram used to identify individual protein components in wound dressing extracts.

FIGS. 14a-d are graphical representations of the amount of fibrinogen, serum albumin, interleukin-1β (IL-1β), and immunoglobulin G (IgG) extracted from an embodiment of a hydrogel composition of the invention and two other comparative dressings after 24 hours of application to partial thickness wounds over the course of healing.

FIGS. 15a-b are graphical representations of the weight fractions of (a) IL-1β and (b) IL-6 in the total proteins extracted from an embodiment of a hydrogel composition of the invention and two other comparative dressings after application to full-thickness wounds. The asterisk indicates significant difference between data at a p<0.05 level of confidence (n≧3; mean±SD).

FIG. 16 is a graphical representation of the distribution of (a) IL-1β and (b) acute phase proteins between wound bed and wound dressing materials after application to 4-day-old full-thickness wounds.

FIG. 17 is a graphical representation of the content of IL-1β against the content of TGF-β1 extracted from wound dressings after application to 4-to-14-day-old full-thickness wounds.

### DETAILED DESCRIPTION

Topical (or cutaneous) inflammation is an immune response triggered by the body as a result of disease, surgery, injury, and/or external irritants, such as toxins, radiation exposure, pathogens, and the like. Topical inflammation typically manifests itself in the form of erythema (redness) and edema (swelling). Undesirable sensations that often accompany topical inflammation include tenderness, burning, itch, and pain. It has now been discovered that the selective removal of certain proteins from a topical site can prevent and/or reduce topical inflammation. More specifically, the selective removal of certain cytokines such as interleukin-1β (IL-1β) and interleukin-6 (IL-6) from a topical site was found to be effective in preventing and/or reducing topical inflammation. The removal of these low-molecular-weight acute phase proteins from the topical site should be selective such that the local concentrations of other proteins, e.g., those having higher molecular weights, at or near the topical site are not substantially altered.

Throughout the description, where compositions are described as having, including, or comprising specific components, or where processes are described as having, including, or comprising specific process steps, it is contemplated that compositions of the present invention also consist essentially of, or consist of, the recited components, and that the processes of the present invention also consist essentially of, or consist of, the recited processing steps.

Additionally, as used herein, the singular forms “a,” “an,” and “the” refer to “one or more” unless the context clearly dictates otherwise. Thus, for example, reference to “a protein” refers not only to a single protein but also to two or more proteins such as a mixture of proteins, and “a polymer” refers not only to one type of polymer but a plurality of polymers such as a blend of polymers and the like. That is, use of the singular includes the plural (and vice versa) unless specifically stated otherwise. In addition, where the use of the term “about” is before a quantitative value, the present teachings also include the specific quantitative value itself (and vice versa) unless specifically stated otherwise.

It also should be understood that the order of steps or order for performing certain actions is immaterial so long as the invention remains operable. Moreover, two or more steps or actions may be conducted simultaneously.

In a first aspect, the invention relates to methods for moderating and/or shortening a topical inflammatory response. The methods can generally include removing one or more cytokines, for example, at least one of IL-1β and IL-6, from a topical site without substantially altering at the topical site a local concentration of one or more plasma proteins, for example, at least one of serum albumin, immunoglobulin G (IgG), and fibrinogen.

As explained in more detail hereinbelow, topical inflammation is characterized by a series of local and systemic reactions, which include the stimulation of various cells to produce cytokines such as IL-1, IL-6, and tumor necrosis factor alpha (TNFα). IL-1 is a particularly important molecule in this series of events. It has been shown that subcutaneous injections of IL-1 could lead to the generation of a local inflammatory infiltrate. See Kupper (1990), J. CLIN. INVEST., 86: 1783-89. Accordingly, the invention provides methods to selectively remove one or more cytokines from a topical site. The one or more cytokines can have a molecular weight of less than about 65 kDa, a molecular weight of less than about 60 kDa, a molecular weight of less than about 55 kDa, a molecular weight of less than about 50 kDa, a molecular weight of less than about 45 kDa, a molecular weight of less than about 40 kDa, a molecular weight of less than about 35 kDa, or a molecular weight of less than about 30 kDa. For example, the one or more cytokines can include at least one of IL-1β and IL-6. The selective removal of at least one of IL-1β and IL-6 from a topical site can reduce the duration and intensity of a topical inflammatory response, as confirmed by the clinical and histological data provided in Examples 1 and 2 below.

In some embodiments, the methods of the invention can include hydrating the topical site. The method also can include contacting the topical site with a physiological buffer. The physiological buffer can include various buffering agents and/or electrolytes including, but not limited to, sodium chloride (NaCl), sodium phosphate (Na₃PO₄, Na₂HPO₄, NaH₂PO₄), potassium sulphate (K₂SO₄), calcium chloride (CaCl₂), MgSO₄, sodium bicarbonate (NaHCO₃), a sodium salt of ethylenediaminetetraacetic acid (EDTA), and the like. Methods of the invention can include restoring the local osmolarity of the topical site, for example, by contacting the topical site with a physiological buffer described above.

In some embodiments, the method includes contacting the topical site with an antimicrobial and/or an anticoagulant. Examples of antimicrobials include, but are not limited to, diazolidinyl urea, quaternary ammonium compounds (e.g., benzalkonium chloride), and various oxidizing agents such as biguanides (e.g., chlorhexidine digluconate), silver compounds (e.g., silver sulphadiazine), and iodine-containing compounds (e.g., iodopropynyl butylcarbamate). Examples of anticoagulants include, but are not limited to, EDTA, heparin, bishydroxycoumarin, and warfarin.

In some embodiments, the topical inflammation is a result of injury or surgery, and the topical site is an open wound. According to these embodiments, the method can include dissolving an effective amount of fibrinogen at the open wound. It has been shown that dehydration of a topical site can trigger conformational changes in a fibrinogen molecule. Such conformational changes transfer the fibrinogen molecule into a proinflammatory state by exposing binding epitopes for the adhesion and accumulation of inflammatory cells. See Hu et al. (2001), BLOOD, 98(4): 1231-38. Soluble fibrinogen is not proinflammatory.

The decrease in local water content at an open wound can lead to hyperosmolarity in skin cells at or near the wound bed. This sudden physiological change can sensitize nerve endings in the dermis and cause pain. By contacting the open wound with a physiological buffer such as the ones described above, the normal osmolarity observed in skin cells can be restored. Therefore, certain embodiments can help alleviate pain and other undesirable sensations that may accompany topical inflammation.

In some embodiments, the method includes applying a hydrogel composition to the topical site. Hydrogel compositions suitable for the practice of the methods of the invention can generally include a protein component and a biocompatible polymer component. Examples of suitable hydrogel compositions are described in U.S. Pat. No. 5,733,563, International Application Publication No. WO 01/74928, and U.S. patent application Ser. No. 10/970,349. The protein component can be crosslinked by the biocompatible polymer component. It has been shown that hydrogel compositions can have excellent hydrating properties. The short-term and long term hydrating effects of an embodiment of a hydrogel composition of the invention on topical sites were demonstrated in co-owned, co-pending U.S. patent application Ser. No. 10/970,349. It also has been shown that certain embodiments of hydrogel compositions of the invention do not induce toxicity or cause irritation to human skin.

The protein component can be obtained from a variety of sources including vegetal sources (e.g., soybean or wheat), animal sources (e.g., milk, egg, or bovine serum), and marine sources (e.g., fish protein or algae). Suitable proteins can include, but are not limited to, bovine serum albumin, human serum albumin, lactalbumin, ovalbumin, soy albumin, pea albumin, hydrolyzed soy protein, hydrolyzed wheat protein, casein, and any combinations thereof. Proteins with abundant charge groups are preferred since they confer excellent water-retaining capacity to the hydrogel compositions.

The biocompatible polymer component of the hydrogel compositions can include various homopolymers, copolymers, or blends of polymers. As used herein, “biocompatible polymer” refers to a natural or synthetic polymer which, alone or in combination with other biocompatible polymers, can form a water-insoluble polymeric layer over the skin that is compatible with the skin as measured by the lack of skin irritation and can be removed from the skin by conventional means, preferably atraumatically. Examples of suitable biocompatible polymers include polyalkylene oxides, polymethacrylates, polyurethanes, cellulosics, polyhydroxyalkyl acrylates, polyesters, and the like.

In certain embodiments, the biocompatible polymer component of the hydrogel compositions includes at least one hydrophilic polymer capable of incorporating and binding relatively high concentrations of water. Examples of such polymers include, but are not limited to, polyethylene glycol (PEG) and its derivatives, e.g., various polyethylene glycols having reactive terminal groups (e.g., carbonates of PEG) or substituents covalently attached to the ethylene carbon atoms of the molecule. When reference to polyethylene glycol or PEG is made herein, it includes such derivatives unless specifically reciting an underivatized PEG. The biocompatible polymer component should have sufficient molecular weight such that after reaction with a protein component, it readily covalently crosslinks the protein component so that the composition gels within a relatively short time. Generally, polymers with weight average molecular weights in the range of about 0.05 Da to about 10×10⁴ Da, or about 0.2 Da to about 3.5×10⁴ Da, or of about 8,000 Da are employed.

The hydrogel compositions can further include buffering agents, antimicrobials, anticoagulants, electrolytes, and other additives, including colorants, fragrance, binders, plasticizers, stabilizers, fire retardants, cosmetics, and moisturizers. For example, the hydrogel compositions can include ethylenediaminetetracetic acid (EDTA), a sodium salt of EDTA, sodium chloride, sodium phosphate, diazolidinyl urea, and/or iodopropynyl butylcarbamate. Other suitable buffering agents, antimicrobial agents, and various additives, as well as methods of incorporating these additives into the hydrogel compositions of the invention, are known by those skilled in the art and are described in co-owned, co-pending U.S. patent application Ser. No. 10/970,349. For example, a desired additive or agent can be incorporated into a hydrogel composition of the invention by immersing the hydrogel composition in an aqueous solution containing the desired additive or agent for an appropriate amount of time.

The hydrogel composition is preferably hydrated when applied to the topical site. For example, the hydrogel composition can include a water content of 50% or more by weight, a water content of 60% or more by weight, a water content of 70% or more by weight, a water content of 80% or more by weight, a water content of 85% or more by weight, a water content of 90% or more by weight, a water content of 95% or more by weight. A fully hydrated hydrogel composition, i.e., a hydrogel composition having a water content of 95% or more by weight, maximizes the hydrating effect of the hydrogel composition, which helps to prevent protein adsorption on the surface of the hydrogel compositions of the invention.

It is well known that the implantation of many polymeric materials can trigger an inflammatory response. See, e.g., Andersson, COMPLEMENT ACTIVATION TRIGGERED BY BIOMATERIAL SURFACES—MECHANISMS AND REGULATION (Acta Universitatis Upsaliensis, Uppsala, Sweden, 2003); Anderson (2001), ANNU. REV. MATER. RES., 31: 81-110). It has been suggested that such inflammatory responses are a result of protein adsorption on the surface of the polymeric materials, particularly the surface adsorption of fibrinogen. See Tang et al. (1996), J. CLIN. INVEST., 97(5): 1329-34. As detailed in Example 4 below, the surface of the hydrogel compositions of the invention can be made resistant to the adsorption of a variety of proteins including fibrinogen, IgG, and serum albumin. Without wishing to be bound by any particular theory, it is believed that the high water content of the hydrogel compositions of the invention (e.g., over 90% by weight) can prevent significant surface adsorption of proteins. Further, without wishing to be bound by any particular theory, it is believed that due to this high water content, the protein component and the biocompatible polymer component of the hydrogel composition can occupy a very small volume within the overall structure of the hydrogel composition. Accordingly, the abundance of water molecules within the hydrogel composition can preclude the formation of a stable surface for any significant protein adsorption. Again without wishing to be bound by any particular theory, even if small amounts of fibrinogen and IgG are adsorbed to the surface of the hydrogel compositions of the invention, their amounts are believed to be insufficient to provide an adequate number of ligands for the adhesion and activation of neutrophils. The hydrating properties of the hydrogel compositions of the invention therefore can help reduce the intensity and duration of a topical inflammatory response.

In some embodiments, the hydrogel composition can include an occlusive membrane to prevent water evaporation. Preferably, the occlusive membrane is oxygen-permeable. In addition, the occlusive membrane can contain perforations such as holes or slits to control the rate of evaporation.

As explained in detail in Example 4 below, the hydrogel compositions of the invention are capable of selectively removing low molecular weight proteins including certain cytokines (e.g., IL-1β and IL-6) from a topical site. Without wishing to be bound by any particular theory, it is believed that this selective removal can be attributed to the unique structural characteristics of the hydrogel compositions of the invention. The macromolecularly crosslinked matrix of polyethylene glycol and protein(s) has been experimentally shown to include relatively large pores. Because of the relatively high porosity, it is believed that small solutes, such as those with a molecular weight less than about 60 kDa, can easily penetrate through the hydrogel-tissue interface and then diffuse within the volume of liquid in the hydrogel network by gradient forces. Unlike IL-1β, which has a molecular weight of about 17 kDa, and IL-6, which has a molecular weight of about 21.5-28 kDa, most proteins including fibrinogen, serum albumin, and IgG, are relatively large. Fibrinogen has a molecular weight of about 240 kDa, while IgG and serum albumin have molecular weights of about 140 kDa and about 66 kDa, respectively. By applying at a topical site a hydrogel composition that selectively absorbs at least one of IL-1β and IL-6, many of the typical local and systemic reactions that are associated with a topical inflammatory response can be largely inhibited if not prevented. Since it has been suggested that by shortening or completely bypassing the inflammatory phase of the wound healing process, wounds can heal potentially without scarring, an aspect of the invention relates to a method of promoting wound healing without scarring. The method can include applying a hydrogel composition of the invention to a topical site immediately after an injury or shortly thereafter, i.e., while the wound healing process is still in the inflammatory phase. For example, the hydrogel composition can be applied within 5 days of an injury, within 4 days of an injury, within 3 days of an injury, within 2 days of an injury, within 24 hours of an injury, within 12 hours of an injury, within 4 hours of an injury, or within one hour of an injury.

To prevent unnecessary injury to the topical site, the hydrogel composition can be non-adherent to the topical site and/or non-adhesive to the surrounding areas of the topical site. These properties can confer certain benefits when the topical site is an open wound. It was previously demonstrated in U.S. Pat. No. 5,733,563, the entire disclosure of which is incorporated by reference herein for all purposes, that the hydrogel composition can possess good mechanical properties, and can conform to the contours of the topical site. To secure the hydrogel composition to the topical site, flexible netting tubes, such as Netelast (Seton Healthcare Group Plc, Oldham, UK), can be used. Other means can be used to secure the hydrogel composition to the topical site, as long as the securing means can be removed, preferably with little or no trauma to the topical site.

To facilitate application on a topical site, the hydrogel compositions can include a backing or support. The backing or support can be or include an occlusive membrane described above. The backing can be polymeric and can be attached to the hydrogel composition with or without the use of an adhesive. As disclosed in co-owned, co-pending U.S. patent application Ser. No. 10/471,463, the entire disclosure of which is incorporated by reference herein for all purposes, a polymeric backing can be adhered to the hydrogel composition by exposing the surface of the polymeric backing to an activated gas. More specifically, a polymeric backing, such as polyethylene terephthalate, can be exposed to plasma of various gases or mixture of gases produced by an excitation source such as microwave and radiofrequency. Gases useful to produce plasma include, but are not limited to, nitrogen, ammonia, oxygen, and various noble gases. A polymeric backing so treated typically can adhere to a hydrogel composition.

In another aspect, the invention relates to a hydrogel composition that can include one or more cytokines. The one or more cytokines can be from an exogenous source (i.e., the cytokines are integrated into the hydrogel composition ex vivo) or an endogenous source (i.e., the cytokines are integrated into the hydrogel composition in vivo). The one or more cytokines can have a molecular weight of less than about 65 kDa, a molecular weight of less than about 60 kDa, a molecular weight of less than about 55 kDa, a molecular weight of less than about 50 kDa, a molecular weight of less than about 45 kDa, a molecular weight of less than about 40 kDa, a molecular weight of less than about 35 kDa, or a molecular weight of less than about 30 kDa. Such a hydrogel composition can be used as a screening tool for determining the efficacy of an anti-inflammatory drug. For example, the hydrogel composition can be applied to a topical site that has been treated with a certain anti-inflammatory drug, where the efficacy of the anti-inflammatory drug can be determined by measuring the amount of cytokines found in the hydrogel composition. The hydrogel composition also can be used as a purification tool for isolating smaller proteins, e.g. cytokines having a molecular weight of less than about 60 kDa, from larger proteins, e.g., plasma proteins such as albumins, globulins and fibrinogen.

Yet another aspect of the invention relates to methods of preparing a hydrogel composition. The method generally includes reacting a protein component with a bifunctional biocompatible polymer. The protein component can include any of the proteins already described. The bifunctional biocompatible polymer can be a bifunctional polyethylene glycol such as a dinitrophenylcarbonyl polyethylene glycol, a dichlorosulfonyl polyethylene glycol, a dichloroacetylsulfonyl polyethylene glycol, a dichlorosulfonyl ethylsulfonyl polyethylene glycol, a diphenylcarbonyl polyethylene glycol, a ditoluenesulfonyl polyethylene glycol, a disuccinimidyl polyethylene glycol, a dimaleimidyl polyethylene glycol, a diisocyanato-polyethylene glycol, or a divinylsulfonamido-polyethylene glycol. International Application Publication No. WO 01/74928 discloses that bifunctional polyethylene glycols such as the ones listed above can be used to form hydrogel compositions by mixing the bifunctional polyethylene glycol with proteins dissolved in aqueous solutions under basic conditions.

The method of preparing a hydrogel composition can include converting a biocompatible polymer into a bifunctional biocompatible polymer. As described in co-owned, co-pending U.S. patent application Ser. No. 10/970,349, the entire disclosure of which is incorporated by reference herein for all purposes, to effect covalent attachment of a PEG to a protein, the hydroxyl end-groups of the polymer can be first converted into reactive functional groups. This process is frequently referred to as “activation” and the resulting bifunctional polyethylene glycol can be described by formula 1:
X—O—(CH₂CH₂O)ₙ-X (1)
where X can be any functional group able to react with the various chemical groups commonly found in proteins, including amino, thiol, hydroxyl, carboxyl and carboxylic groups; and n can vary from about 45 to about 800, which corresponds to commercial PEG of molecular weight ranging from about 2,000 to about 35,000 Daltons. The activation step can be conducted in solvent or in a solvent-free environment as detailed in co-owned, co-pending U.S. patent application Ser. Nos. 10/487,392 and 11/071,877, the entire disclosures of which are incorporated by reference herein for all purposes.

U.S. patent application Ser. No. 10/487,392 describes a method for preparing activated PEGs with p-nitrophenyl chloroformate. The method involves a reaction carried out at room temperature using an aprotic solvent, such as methylene chloride (CH₂Cl₂), in the presence of a catalyst, such as dimethylaminopyridine (DMAP). U.S. patent application Ser. No. 11/071,877 describes alternative methods of activating PEG, e.g., by reacting molten PEG with an activator in a solvent-free environment. Additionally, commercial PEG-dinitrophenyl carbonates suitable for preparing hydrogel compositions of the present invention are available, and can be purchased from Nektar Therapeutics (Huntsville, Ala.).

Hydrogel compositions can have a variety of desirable properties. As described in co-owned, co-pending U.S. patent application Ser. No. 10/970,349, hydrogel compositions can be imparted with desirable pharmaceutical activities, including antimicrobial activities, by incorporating suitable pharmaceutically active agents into the hydrogel compositions. By preventing inflammation and infection, the methods of the invention can accelerate and/or improve the healing of open wounds, as seen in, for example, a faster rate of reepithelialization and wound closure, and the absence of scarring.

The following examples are provided to illustrate further and to facilitate the understanding of the invention and are not intended to limit the invention in any way.

### EXAMPLE 1

Clinical Observations of Topical Inflammation and Wound Infections

Animal studies were performed to evaluate the intensity and duration of topical inflammation associated with the healing of partial thickness wounds as well as full thickness wounds as modulated by five different types of wound dressings.

Procedures

In the partial-thickness wounds study, six young domestic pigs weighing about 15 kg to about 18 kg were conditioned for at least 2-3 days prior to the experiment. Animals were fed a commercial growing diet and housed individually at a controlled temperature ranging from about 20° C. to about 25° C. The experimental protocol was approved by the Ethical Committee of the Department of veterinary medicine of the University of Montreal, and the animals were handled according to the “Guide for the Care and Use of Laboratory Animals.” Skin of the animals was shaved with a hair clipper and washed with neutral soap. No aseptic solution was applied on the skin.

On day 0, four 5 cm×10 cm rectangular regions (treatment regions A, B, C and D) were drawn on the back of each pig. Within each region, two partial thickness wounds were surgically created with a Padgett electric dermatome (Padgett Instruments, Inc., Plainsboro, N.J.) while the animals were under general anesthesia. Each wound measured about 3 cm×2 cm, with a thickness of about 300 μm.

Immediately after surgery, the following types of wound dressings were applied to the various wounds: i) hydrogel compositions prepared according to the method described below (referred herein as “BioAquaCare™”); ii) 2ND SKIN® Moist Bum Pads (Spenco Medical Corporation, Waco, Tex., referred herein as “2^{nd} Skin®”); iii) NEXCARE™ TEGADERM™ Transparent Dressings (3M, St. Paul, Minn.; referred herein as “Tegaderm™”); and iv) conventional dry gauze dressings (referred herein as “dry gauze”). BioAquaCare™, 2^{nd} Skin®, and dry gauze were applied under occlusive conditions using Tegaderm™ as a secondary dressing. Dressings were changed every day until complete wound closure was observed. The criteria used to determine whether a wound had achieved complete closure was similar to the criteria used to determine whether a graft donor site is ready to be re-used for future skin grafting, i.e., the skin should be healthy and should have a sufficient thickness to provide a useful graft without causing deeper wounds. Table 1 summarizes the treatment plan for each of the pigs.

TABLE 1
Types of wound dressings applied on the various
partial thickness wounds on the tested animals.

| | TREATMENT REGIONS | | | |
|---|---|---|---|---|
| | A | B | C | D |
| PIG 1 | BioAqua- | BioAqua- | BioAqua- | BioAqua- |
| | Care ™ | Care ™ | Care ™ | Care ™ |
| PIG 2 | BioAqua- | BioAqua- | BioAqua- | BioAqua- |
| | Care ™ | Care ™ | Care ™ | Care ™ |
| PIG 3 | 2^{nd} Skin ® | 2^{nd} Skin ® | Tegaderm ™ | Tegaderm ™ |
| PIG 4 | 2^{nd} Skin ® | 2^{nd} Skin ® | Tegaderm ™ | Tegaderm ™ |
| PIG 5 | Dry gauze | Dry gauze | Dry gauze | Dry gauze |
| PIG 6 | Dry gauze | Dry gauze | Dry gauze | Dry gauze |

In the full-thickness wounds study, BioAquaCare™, 2^{nd} Skin®, Urgotul® dressing (Laboratoires Urgo, Chenôve, France, referred herein as “Urgotul®”), and dry gauze were tested on 9 pigs. All procedures were performed in accordance with the requirements outlined in the “Guide for the Care and Use of Laboratory Animals.” This study was approved by the Ethical Committee of the Department of veterinary medicine of the University of Montreal. Prior to surgery, the skin of the animals was shaved and washed. All surgeries on the animals were conducted under general anesthesia as described above in connection with the partial-thickness wounds study.

Four full-thickness dorsal wounds reaching muscular fascia were created in each animal using a biopsy punch of 25 mm in diameter. Wound dressings were applied immediately after wounding, and the animals were bandaged with a combination of 3M™ Vetrap™ bandaging tape (3M, St. Paul, Minn.) and elastomer bandages. Wound dressings were changed every two days until complete wound closure.

At each dressing change, each wound was visually inspected for the following visible signs of inflammation, infection, or healing: i) wound fluid interaction; ii) erythema (redness); iii) edema (swelling); iv) infection (purulent fluid, heat, and/or foul odor); v) blood clot; vi) fibrin; vii) scab around the wound; viii) scab on the wound; and ix) presence of scar. More particularly, the frequency of the occurrence of these phenomena was noted and expressed as a percentage calculated using the formula below:
[Number of times a visible sign was observed with a particular treatment plan/(Number of wounds treated by that treatment plan * Number of observations made between day 0 and complete wound closure)]*100%

The rate of wound closure was determined by the reduction of the wound size with the help of planimetry and digital photography. At each dressing change, the boundaries of the wounds were traced using a template the size of the initial injury and changes in the wound size were recorded.

Reepithelialization of the wounds also was evaluated using a semi-quantitative scoring system where a score of 1=no reepithelialization; a score of 2=<25% reepithelialization; a score of 3=26-50% reepithelialization; a score of 4=51-75% reepithelialization; a score of 5=76-87% reepithelialization; a score between 5.1 and 5.9=88-99% reepithelialization; and a score of 6=100% reepithelialization were used.

In addition, standardized photographs were taken with a digital camera and a 35 mm camera at a right angle to the wound surface at defined time points depending on the particular treatment plan.

Materials

BioAquaCare™ was prepared as follows. An aqueous solution of activated polyethylene glycol (PEG) was mixed with an equal volume of a soy protein solution. The resultant mixture was cast between two films to give a hydrogel with a thickness of about 1.8 mm and cut to a dimension of about 8 cm by 20 cm. After polymerization, the hydrogel was incubated in a buffered solution to remove by-products and unreacted PEG and soy protein. The purified hydrogel was submerged in a phosphate-buffered saline solution containing ethylenediaminetetraacetic acid (EDTA) (0.9 wt. % sodium chloride, 0.2 wt. % EDTA, and 0.16 wt. % sodium phosphate monobasic) and preservatives at pH 5.5 for two hours.

The other comparative dressings were used as received. 2^{nd} Skin® is a hydrogel material made of a modified graft copolymer of methyl vinyl ether and maleic acid (see U.S. Pat. No. 5,393,798). Tegaderm™ is a semi-permeable adhesive dressing made of polyurethane. Urgotul® is a non-occlusive dressing that includes a polyester mesh impregnated with hydrocolloid particles dispersed in a petroleum jelly matrix. The mesh is made up of 100% continuous non-deformable polyester filaments with a mesh opening diameter of 0.5 mm. The dry gauze dressings used are conventional woven dressings made of cotton.

Results

A. Partial-Thickness Wounds

It was observed that Pig 1 had deeper wounds than the other five pigs (histological pictures revealed the presence of dermal papillae). Accordingly, results obtained from Pig 1 were excluded unless otherwise specified.

TABLE 2
Frequency of occurrence of certain visible signs of inflammation,
infection, or healing from day 0 to complete wound closure.

| Visible sign of | Type of primary wound dressing applied | | | |
|---|---|---|---|---|
| inflammation/ | BioAqua- | | Tega- | Dry |
| infection/healing | Care ™ | 2^{nd} Skin ® | derm ™ | gauze |
| Erythema | 11% | 33% | 50% | 38% |
| Edema | 0% | 17% | 17% | 25% |
| Infection sign | 0% | 17% | 33% | 0% |
| Blood clot | 0% | 17% | 0% | 0% |
| Fibrin | 22% | 17% | 17% | 0% |
| Scab around the | 44% | 83% | 83% | 25% |
| wound | | | | |
| Scab on the wound | 44% | 33% | 50% | 63% |

Table 2 above shows the frequency of occurrence of certain visible signs of inflammation, infection, or healing observed during the study (day 0 to complete wound closure). Data from Pig 1 are included in the results presented in Table 2.

It was observed with BioAquaCare™, 2^{nd} Skin®, and Tegaderm™, that each of these three types of dressings was able to exchange fluids with the wound bed to some degree. Exchange of wound fluids was confirmed by the change of color in the dressing (from transparent or opaque to a spectrum of yellowish/brownish/reddish colors) and signs of cleansing of the wound bed (reduction of cellular debris). By comparison, wounds treated with dry gauze appeared very dry, and had a crusty layer of desiccated residues of wound fluid on the wound. It was concluded that due to their hyper-absorbency and permeability properties, the dry gauze dressings could only absorb wound fluid unilaterally with virtually no fluid exchange with the wound bed.

The inflammatory phase associated with wound healing typically begins within hours of the insult, peaking at 24-48 hours after the injury, and lasting until the fifth or sixth day into the wound healing process. Common signs of inflammation include redness (erythema), swelling (edema), pain, and heat. Among the four types of wound dressings studied, wounds treated with BioAquaCare™ (including the thicker wounds on Pig 1) exhibited minimal, if not the complete lack of, signs of inflammation, as reflected in the frequency percentages related to erythema (11%) and edema (0%) presented in Table 2. By comparison, erythema and edema were more commonly observed with wounds treated with 2^{nd} Skin®, Tegaderm™, and dry gauze dressings (2^{nd} Skin®: erythema—33%, edema—17%; Tegaderm™: erythema—50%, edema—17%; dry gauze: erythema—38%, edema—25%). During the first 6 days of the treatment, these wounds tend to have a general red surface, sometimes accompanied by more intense red dots underneath the surface. It was observed that wounds treated with Tegaderm™ had the most swelling.

Referring again to Table 2, wounds treated with BioAquaCare™ did not show any clinical signs of infection. Specifically, no purulent fluid, heat, or foul odor was observed with or detected from any of the wounds treated with BioAquaCare™ on either Pig 1 or Pig 2. By comparison, wounds treated with 2^{nd} Skin® and Tegaderm™ dressings exhibited apparent signs of infection (2^{nd} Skin®−17%; Tegaderm™—33%). The most severe infections were observed on wounds treated with Tegaderm™. Tegaderm™ was observed to be ineffective in absorbing wound fluids, which led to an accumulation of wound fluid on the wound bed, and provided a wet environment favoring the proliferation of pathogenic bacteria. None of the wounds treated with dry gauze was observed to be infected.

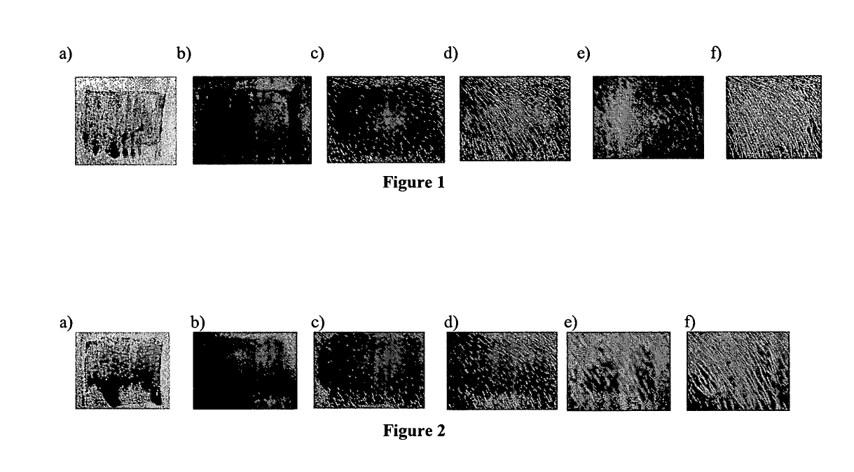

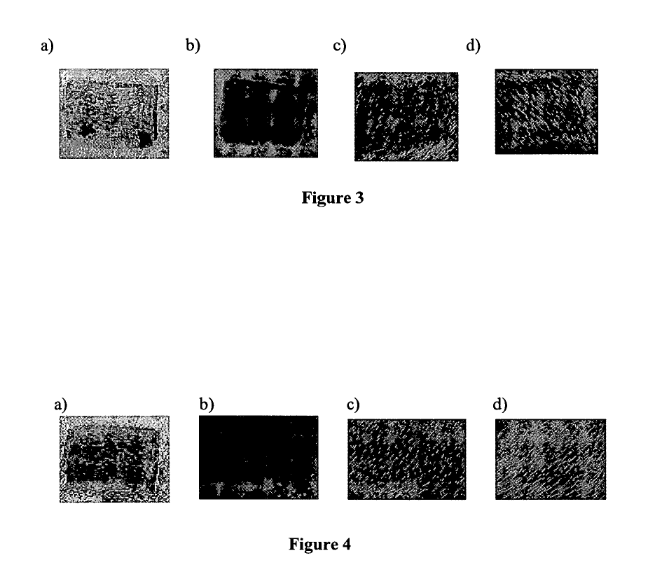

Further comparisons regarding the efficacy of the different wound dressings can be made by examining the standardized photographs of the various wounds taken during the study. FIGS. 1 to 6 show the healing process of the various wounds treated with the four types of dressings. FIG. 1 shows the healing process of a representative wound on Pig 1 treated with BioAquaCare™. The photographs were taken on day 0 (a), day 2 (b), day 4 (c), day 6 (d), day 9 (e), and day 11 (f), respectively. FIG. 2 shows a second wound on Pig 1 also treated with BioAquaCare™. The photographs were taken on day 0 (a), day 2 (b), day 4 (c), day 6 (d), day 9 (e), and day 11 (f), respectively. FIG. 3 shows the healing process of a representative wound on Pig 2 treated with BioAquaCare on day 0 (a), day 2 (b), day 4 (c), and day 6 (d), respectively. FIG. 4 shows the healing process of a representative wound on Pig 3 treated with 2^{nd} Skin® on day 0 (a), day 2 (b), day 4 (c), and day 6 (d), respectively. FIG. 4 shows the healing process of a representative wound on Pig 4 treated with Tegaderm™ on day 0 (a), day 2 (b), day 4 (c), day 6 (d) and day 7 (e), respectively, after scabs were removed from the wound on days 4 and 6. FIG. 5 shows the healing process of a representative wound on Pig 5 treated with dry gauze on on day 0 (a), day 2 (b), day 4 (c), day 6 (d) and day 7 (e), respectively, after scabs were removed from the wound on days 4 and 6.

As mentioned above, the wounds on Pig 1 were inadvertently made deeper than the wounds made on the other five pigs. Despite the deeper wounds, complete wound closure was observed on day 11 without scarring, and the new epidermis had a satisfactory thickness (FIGS. 1a-f). Referring to FIGS. 3a-d, it can be observed that the partial thickness wounds on Pig 2 showed no signs of inflammation or infection throughout the course of the study. The surfaces of the wounds were more than 50% colonized by a neo-synthesized epidermis on day 4, and about 76% to about 87% reepithelialized by day 6. Wound closure was completed on day 6 without scarring, and the color of the wound site was similar to the surrounding normal skin. No blood clot was observed during the entire healing process, and few scabs were observed around or on the wounds.

Referring to FIGS. 4a-d, wounds treated with 2^{nd} Skin® were reepithelialized after 6 days of treatment. Although wounds treated with 2^{nd} Skin® were able to heal at a rate comparable to those treated with BioAquaCare™, they showed signs of both inflammation and infection during the course of healing (see, e.g., FIG. 4c).

With reference to FIGS. 5a-e, wounds treated with Tegaderm™ were healing more slowly compared to wounds treated with either BioAquaCare™ or 2^{nd} Skin®. It took an average of 7 days for wounds treated with Tegaderm™ to close completely. Some of the wounds treated with Tegaderm™ showed signs of intense inflammation until the sixth day of treatment. Infections were also quite common. A foul odor was detectable, and a purulent exudate could be observed on most of the wounds. It was observed that a large portion of the wounds were covered with scabs, which had to be removed to appreciate the rate of reepithelialization. Scabs were removed very carefully to avoid ripping the repaired skin underneath.

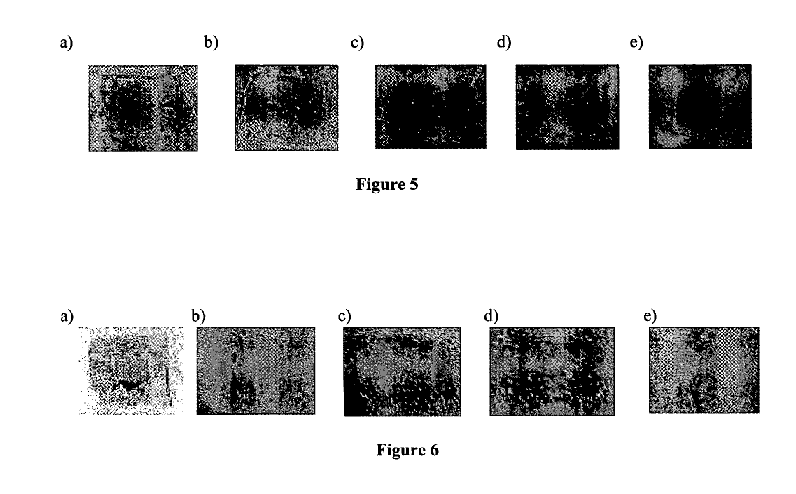

With reference to FIGS. 6a-e, wounds treated with dry gauze (Pig 5 and Pig 6) were the slowest to heal among the four types of treatment studied. It took 9 days for these wounds to achieve complete wound closure. Many inflammatory signs were observed with these wounds during their healing process. In some cases, intense inflammation was observed. For example, FIG. 6c shows that a wound treated by dry gauze had significant edema. Additionally, it was observed that scabs covered most of the wounds treated with dry gauze. Excessive scab formation can limit the movement and reorganization of epithelial cells, which can lead to prolonged wound healing as observed with these wounds.

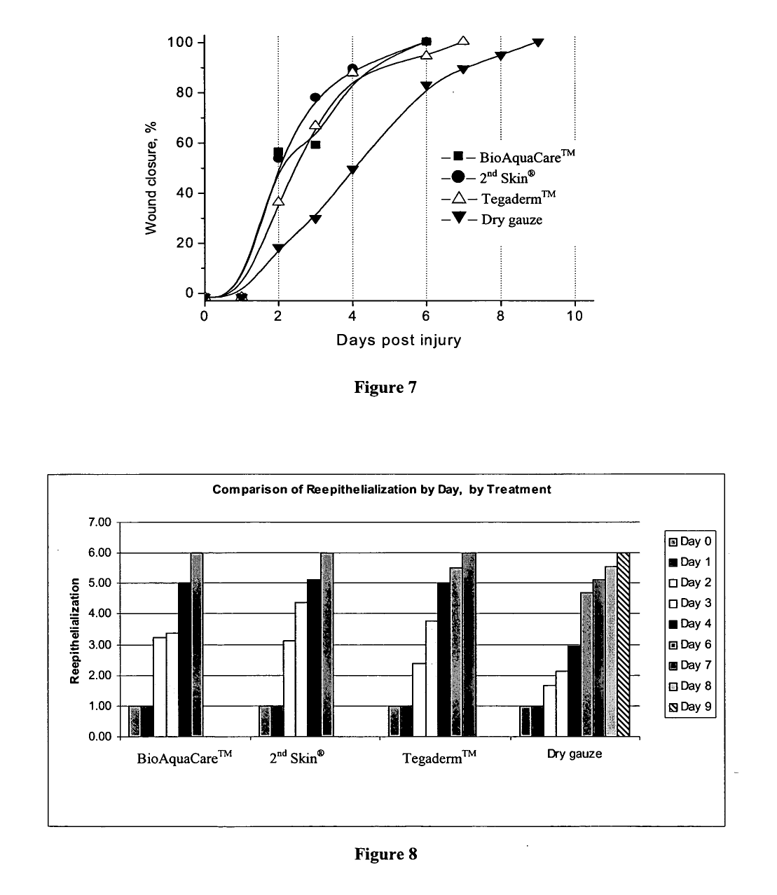

FIGS. 7 and 8 show the rate of reepithelialization in relation to each type of dressing. Results from Pig 1 were excluded because of the anomaly in wound thickness. In FIG. 7, the rate of reepithelialization was estimated in terms of percentage. In FIG. 8, the rate of reepithelialization was evaluated using the scoring scale of 1 to 6 explained previously in the procedures section above. As previously discussed, wounds treated with BioAquaCare™ and 2^{nd} Skin® were reepithelialized after 6 days of treatment, compared to 7 days for wounds treated with Tegaderm™, and 9 days for wounds treated with dry gauze.

B. Full-Thickness Wounds

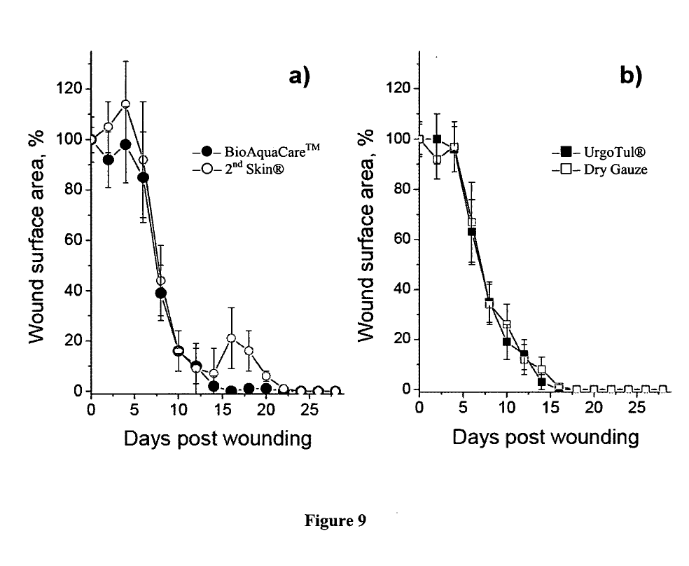

Referring to FIGS. 9a-b, irrespective of the dressing used, all of the full-thickness wounds were substantially closed 28-30 days after surgery. In some instances, however, edema, erythema and/or wound infection were observed, which impeded the normal wound healing process. For example, edema was observed on wounds covered with 2^{nd} Skin®, which resulted in a significant expansion of the wounds at the beginning of the wound healing process. Also, occasional re-opening of the wounds due to bacterial infection was observed at the later stages of wound healing (Figure a, 2^{nd} Skin®; days 15-18).

It was shown by the results obtained in these studies that moist wound dressings such as BioAquaCare™ and 2^{nd} Skin® were believed to accelerate reepithelialization by preventing desiccation of denuded dermis or deeper tissues, thereby allowing faster migration of epidermal cells, e.g., keratinocytes, to the wound surface. Excessive or ineffective absorption of wound fluids, as illustrated by wounds treated with dry gauze and Tegaderm™, respectively, could both prolong and intensify inflammation, and delay complete reepithelialization. Compared to 2^{nd} Skin®, BioAquaCare™ was demonstrated to offer the additional advantages of reducing both the intensity and duration of the inflammation phase, and preventing infection as well as scar formation.

### EXAMPLE 2

Histological Evidence of Topical Inflammation

Procedures

To evaluate how treatment with different types of wound dressing affects topical inflammation at a cellular level, dermal specimens including the wound sites studied in Example 1 were obtained.

Animals were sacrificed at predetermined time points by an overdose of sodium pentobarbital. Immediately after sacrifice, dorsal skin of each pig was removed and fixed in 10% neutral buffered formalin. After 2 days, the fixed tissues were embedded in paraffin blocks. Wounded areas of the skin were identified and sliced at 5 μm. Hematoxylin/eosin staining was used for general observation, and Masson's trichrome coloration was used for analysis of collagen organization.

Histological assessments and analyses were made using a Leica DM4000B Microscope (Leica Microsystems, Cambridge, United Kingdom). Histological photographs were taken with a Leica Digital FireWire Camera connected to a Leica Q550 Imaging Workstation (both from Leica Microsystems, Cambridge, United Kingdom).

Results

A. Partial-Thickness Wounds

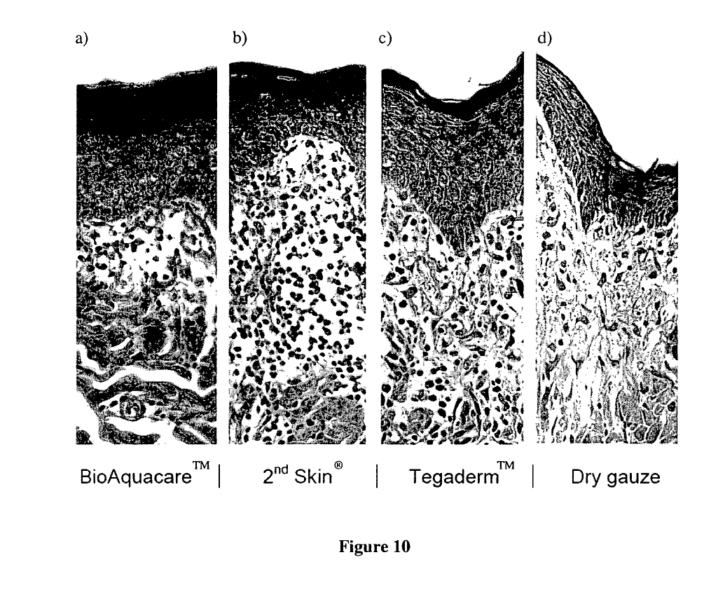

Low level of inflammation in the BioAquacare™-treated wounds was confirmed by histological analysis of the tissue (FIG. 10). Cellular composition of the wounds re-epithelialized under moist occlusive conditions provided by BioAquacare™ was very similar to that of normal skin, with well-developed stratum corneum and epithelial layer. Collagen was more or less completely remodeled to form bundle texture. At the same time, moderate to severe inflammation was observed in the wounds treated with 2^{nd} Skin® and Tegaderm™ (FIG. 10). These products induced intense host response as evidenced by the accumulation of a mixed inflammatory infiltrate in the vicinity of the newly formed epithelium. Inflammation also was observed with the use of dry gauze, albeit to a lesser extent (FIG. 10). Such inflammatory response eventually subsided and largely terminated 4-5 days after complete re-epithelialization.

The results of this histological study confirmed that partial thickness wounds treated with BioAquaCare™ were able to heal without any significant inflammation. This lack of inflammatory response was observed clinically in Example 1 and was confirmed by the histological evidence presented in this example.

B. Full-Thickness Wounds

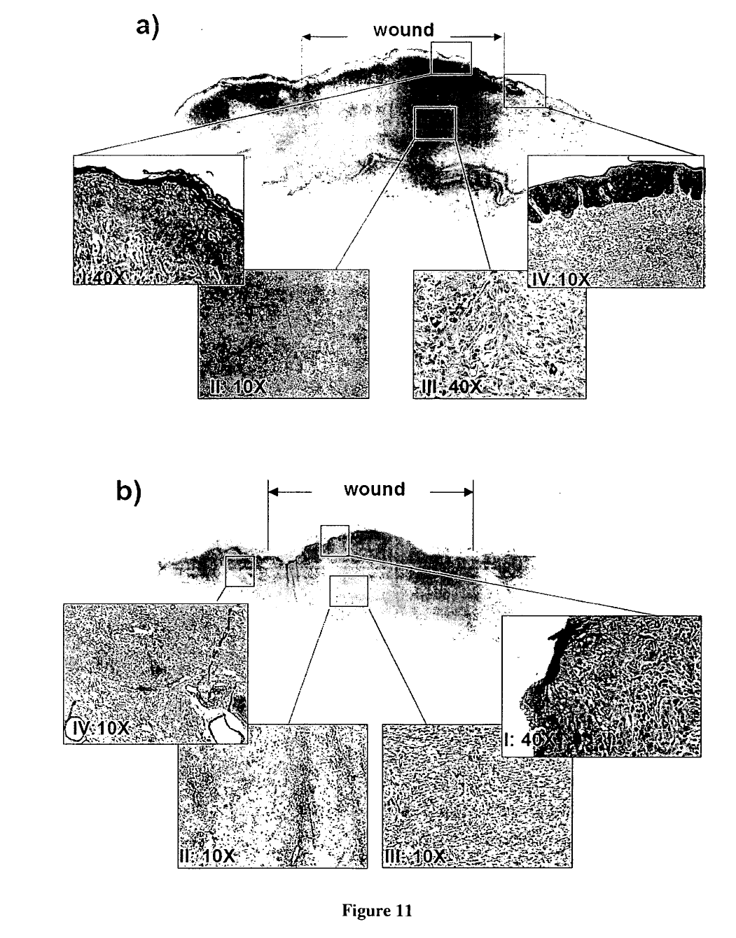

Histological examination of the wounds revealed noticeable difference in the biological response to the injuries and/or wound dressing application (FIG. 11). After 10 days of treatment, the wounds treated with BioAquaCare™ were completely filled with granulation tissue and populated with fibroblasts (FIG. 11a). Newly synthesized collagen fibers, albeit not fully remodeled, were deposited in a basket-weave pattern, surprisingly similar to that of intact dermis. On day 10, the wounds were 75-80% re-epithelialized, and neo-epidermis was found to be well-stratified and keratinized (FIG. 11a). Healing of the wounds treated with BioAquaCare™ appeared to have taken place with minimal inflammation, as evidenced by the relatively low accumulation of inflammatory cells in the vicinity of the injuries.

A very different cellular response was observed with the gauze-covered wounds, where severe inflammation was detected in the subcutaneous and perivascular tissues, as well as in the periphery of the injuries (FIG. 11b). Starting from day 10, acute inflammation began to subside, but cessation of inflammation was observed only in the 20-day old wounds. After three weeks, immature cell-dense granulation tissues with poorly organized collagen deposits were still present in the upper dermal layer.

### EXAMPLE 3

Microbiological Evidence of Wound Infections

Skin is covered with micro-organisms. Bacteria such as Staphylococcus epidermidis, corynebacteria, brevibacteria, and other coagulase-negative staphylococci form part of the normal skin flora. Although wound flora is usually different from normal skin flora, microbial colonization of the wound by itself generally does not lead to wound infections. It is only when host defenses are no longer able to maintain a manageable bioburden, whether in terms of species or number, that critical colonization results. Once this happens, the wound will most likely become infected if left untreated. Examples of common pathogens responsible for wound infections include Staphylococcus aureus, beta-hemolytic streptococci (S. pyogenes, S. agalaciae), Escherichia coli, Proteus, anaerobes, Pseudomonas, Acinetobacter, and Stenotrophomonas (Xanthomonas).

Procedures

To monitor changes in wound flora over the course of the wound healing process, a microbiological study was performed to identify the microorganisms found on partial thickness wounds as they were treated with BioAquaCare™, 2^{nd} Skin®, Tegaderm™, and dry gauze dressings. Concomitantly with the study described in Example 1, swab cultures were obtained from each wound at each dressing change using a one-point swab collection technique. Specifically, Staphylococcus Aureus were cultured on mannitol salt agar, while Streptococcus agar was used to culture both alpha- and beta-hemolytic streptococcus. Enterobacter species were cultured on eosin methylene blue (EMB) agar.

Results

Table 3 below summarizes the types of pathogens observed on partial thickness wounds treated with BioAquaCare™, 2^{nd} Skin®, Tegaderm™, and dry gauze dressings over the course of healing. Data regarding treatment with 2^{nd} Skin® and Tegaderm™ beyond day 6 are not available due to the death of the animals.

Referring to Table 3, Staphylococcus aureus was found to be present on day 2 on wounds treated with BioAquaCare™. However, further growth of the bacteria appeared to be inhibited by the continued treatment with BioAquaCare™, as the presence of Staphylococcus aureus was not detected beyond the second day of treatment.

TABLE 3
Pathogenic species identified on partial thickness wounds treated
with BioAquaCare ™, 2^{nd} Skin ®, Tegaderm ™,
and dry gauze dressings over the course of healing.

| | | BioAqua- | 2nd | Tega- | Dry |
|---|---|---|---|---|---|
| Day | Pathogen | Care ™ | Skin ® | derm ™ | gauze |
| Day 2 | Staphylococcus | X | | | |
| | aureus | | | | |
| | B-haemolytic | | | X | |
| | Streptococci | | | | |
| | Group A or B | | | | |
| | Pseudomonas | | X | | X |
| | aeruginosa | | | | |
| Day 3 | B-haemolytic | | | | X |
| | Streptococci | | | | |
| | Group A or B | | | | |
| Day 6 | Staphylococcus | | X | X | X |
| | aureus and | | | | |
| | species | | | | |
| | Pseudomonas | | X | X | X |
| | aeruginosa | | | | |
| Day 8 | Staphylococcus | | Not | Not | X |
| | aureus | | measured | measured | |
| | Pseudomonas | | Not | Not | X |
| | species | | measured | measured | |
| | B-haemolytic | | Not | Not | X |
| | Streptococci | | measured | measured | |
| | Group A or B | | | | |

The visible signs of infection observed on wounds treated with 2^{nd} Skin® and Tegaderm™ were confirmed by the microbiological evidence presented in this study. A high density of bacterial colonization, especially late in the course of the treatment, was observed on wounds treated with both 2^{nd} Skin® and Tegaderm™.

It also was observed that pathogenic microorganisms were present on wounds treated with dry gauze throughout the course of the study. The species of pathogens observed follow the natural evolution of an infected wound.

The results of this microbiological study confirmed that partial thickness wounds treated with BioAquaCare™ were able to heal without any significant infection. The lack of wound infection was observed clinically in Example 1 and was confirmed by the microbiological evidence presented in this Example.

The combined results from Examples 1-3 conclude that treatment with BioAquaCare™ reduced and/or prevented inflammation and infection, which in turn accelerated the healing process of both partial- and full-thickness wounds. Clinical observations from Example 1 are well supported by the histological evidence presented in Example 2 and the microbiological evidence presented in Example 3. None of the other four types of dressings studied was able to induce healing at a comparable rate to BioAquaCare™ while controlling inflammation and infection as effectively as BioAquaCare™.

### EXAMPLE 4

Protein Adsorption/Absorption by Different Dressings During the Inflammatory Phase

While the application of wound dressings aims to promote wound healing, the surfaces of most wound dressings are expected to adsorb at least some proteins from the wound site. Such protein adsorption can trigger inflammation as it has been observed with a lot of implanted biomaterials. It has been suggested that acute inflammatory responses to biomaterials require the precedent adsorption of fibrinogen. See Tang et al. (1996), J. CLIN. INVEST., 97(5): 1329-1334.

To study protein adsorption/absorption by the different dressings during the inflammatory phase, wound dressings used in Example 1 were collected immediately after their application and subjected to protein extraction procedures and various assays. Tegaderm™ dressings were not collected as they were found to be ineffective in absorbing wound fluids. It was observed that all of the BioAquaCare™, 2^{nd} Skin®, and dry gauze dressings collected from Example 1 were virtually undamaged, and appeared fully swollen, indicating good water-binding capacity of the material.

Statistical analysis (n≧3) was performed on all quantitative data described below using an analysis of variance (ANOVA) to a significance level of p≧0.01 or p≧0.05. Results presented in the following tables and figures include the means and the standard deviation. Content of cytokines and growth factors is expressed in ng of the cytokine or growth factor in 1 g of proteins extracted from the dressing (ng/g).

Part A. Total Protein Quantification

Procedures

This part of the study aims to determine the total amount of proteins adsorbed by the different dressings. The first step of the protein extraction procedures involved immersing the dressings in 10 mL of phosphate-buffered saline (PBS) at room temperature or at 4° C. for two hours upon gentle agitation. In some cases, the extraction mixture media were sonicated for 20 seconds to facilitate protein dissolution.

After 3 hours of extraction, aspirated liquid containing wound dressing extracts was centrifuged at 3000 rpm for 15 minutes at 4° C. using a Sorvall® RT-7 centrifuge (available from Kendro Laboratory Products, Asheville, N.C.). The supernatants obtained after centrifugation were kept frozen at −25° C. until further analysis. In some cases, a second extraction of the dressings was performed in 10 mL of PBS. It was observed that none of the dressings showed signs of degradation after 24 hours of extraction. Wound protein extracts were prepared by washing the wound bed with 10 mL of sterile PBS for several minutes, as proposed by Saymen et al. (1973), Appl. Microbiol. 25: 921-934. The samples of wound protein extracts were filtered sterilely and kept frozen.

A bicinchoninic acid (BCA) protein assay was performed to determine the total amount of proteins in the wound dressing extraction solution. The BCA protein assay is a detergent-compatible formulation based on bicinchoninic acid for the colorimetric detection and quantification of total protein. This method combines the well-known reduction of Cu⁺² to Cu⁺¹ by protein in an alkaline medium (the biuret reaction) with the highly sensitive and selective colorimetric detection of the cuprous cation (Cu⁺¹) using a reagent containing bicinchoninic acid. See Moseley et al. (2004), J. DERMATOL., 150: 401-413. The purple-colored reaction product of this assay is formed by the chelation of two molecules of BCA with one cuprous ion. This water-soluble complex exhibits a strong absorbance at 562 nm that is linear with increasing protein concentrations over a broad working range of 20 μg/ml to 2,000 μg/ml.

Samples of wound dressing extraction solution (typically 5 or 10 μL) were transferred into a 96-well plate and diluted with 90 μL of PBS. Similarly, a stock solution of BSA (0.5 mg/mL) was diluted in PBS to obtain final concentrations of 0.050, 0.100, 0.250, 0.375, and 0.500 mg/mL. A 100-μL portion of BCA reagent was added to each well and the reaction proceeded for 20 minutes at room temperature. After incubation, absorbance readings at 540 nm were obtained and used to calculate the total protein content of each extract samples. The total protein content was expressed as the weight of proteins recovered from each gram of the wound dressing.

Results

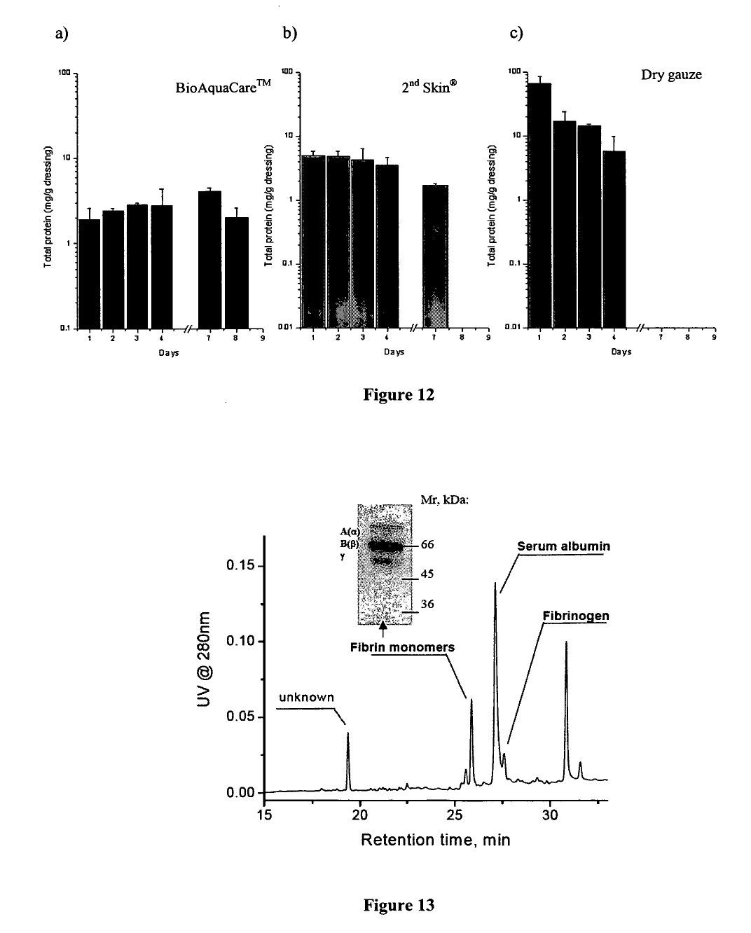

FIG. 12 compares the total protein content recovered from BioAquaCare™, 2^{nd} Skin®, and dry gauze dressings that were collected throughout the healing process of the partial thickness wounds described in Example 1. No protein extraction was possible with the dry gauze dressings collected on day 7 and beyond, since the dry gauze dressings had completely dried up the wound beds by day 6, and a layer of crust was observed on the wounds themselves.

Referring to FIG. 12, it can be seen that among the studied wound dressings, BioAquaCare™ was the least adsorptive for wound fluid proteins (about 1.4-2.0 mg per gram of dressing versus about 4.0-5.0 mg/g in the case of 2^{nd} Skin® and about 60.0 mg/g for dry gauze).

It was also observed that while the protein content recovered from 2^{nd} Skin® and dry gauze dressings decreased over time during the studied period (from about 65 mg/g on day 1 to about 4 mg/g on day 4 for dry gauze dressings), the protein content recovered from BioAquaCare™ steadily increased over time (from about 1.9 mg/g on day 1 to about 4.0 mg/g on day 7). After the first 7 days, the total protein level recovered from BioAquaCare™ started to decrease.

Part B. Quantification of Interleukin-1β, Immunoglobulin G, Serum Albumin and Fibrinogen

Fibrinogen, interleukin-1β (IL-1β), and immunoglobulin G (IgG) are some of the most important proteins involved in the wound healing process. Serum albumins account for more than 50% of the total serum proteins and are responsible for maintaining the colloidal osmotic pressure of the proteins in plasma. This part of the study aims to determine the respective amount of fibrinogen, serum albumin, IL-1β, and IgG, that was adsorbed by the different dressings.

Procedures

Component composition of proteins was studied by means of reverse-phase high-performance liquid chromatography (RP-HPLC). RP-HPLC was performed in the gradient mode on a system consisting of a type 600E pump, a type 776 autosampler and a type 996 Photo Diode Array detector, all obtained from Waters (Milford, Mass.). The column used was an Ace C8 (300×4.6 mm I.D., particle size of 5 μm) silica-based column thermostabilized at 40° C. The mobile phase composed of 0.1% trifluoroacetic acid (TFA) in water (eluent system A) and 80% (v/v) acetonitrile in 0.075% TFA (eluent system B) was pumped at a flow rate of 1.0 mL/min and the column effluent was measured within the wavelength range of 210-300 nin at a 4.8 nm resolution. The elution was carried out with a linear gradient of B from 0 to 90% for 30 minutes followed by the isocratic elution in 90% of B for 5 minutes.

Before chromatography, mobile phases were filtered through membrane filters (pore diameter of 0.22 μm) (Millipore, Billerica, Mass.) and degassed for 20 min.

Quantification of IgG was performed by Protein A affinity chromatography on a HiTrap™ Protein A HP column from Amersham Biosciences (Uppsala, Sweden). Specifically, IgG was first isolated from wound fluid extracts by using an HPLC column packed in-house with Protein A Sepharose CL-4 (Amersham Pharmacia Biotech, Piscataway, N.J.). Dry powder of the affinity sorbent (0.500 g) was reconstituted in 2 mL of PBS (pH 7.4), and the slurry obtained was transferred to the HPLC column (5.0 cm×4.6 mm i.d.). The column was connected to the above-described Waters chromatography system (Milford, Mass., USA) and was equilibrated with PBS, washed and used for IgG extraction, according to the following protocol: injection volume 250 μL; flow rate—2.0 ml/min; isocratic elution with PBS for 5 min, followed by 2-min gradient from PBS to 25 mM phosphoric acid and final elution in 25 mM phosphoric acid. Detection of the eluates was carried out at 280 nm.

Quantification of IL-1β, IL-6 and TGF-β1 was performed by immunoassay with ELISA kits purchased from R&D Systems Inc. (Minneapolis, Minn.) and used according to manufacturer's instructions. The amount of the four specific protein components of interest was expressed as the weight of the proteins (in mg for serum albumin, IgG, and fibrinogen, and in μg for IL-1β) per gram of the dressing from which the proteins were extracted.

Results

Using RP-HPLC, serum albumin, fibrinogen, fibrin monomers and IgG were identified as the most abundant components of the wound dressing extracts. The HPLC chromatogram is shown in FIG. 13. The peaks of serum albumin and fibrinogen were identified by comparing the elution profiles of wound fluid components with the position of peaks obtained for pure proteins. A component eluted at approximately 26 minutes was attributed to modified molecules of fibrinogen, i.e., fibrin monomers. This component migrated similarly to intact fibrinogen upon SDS-PAGE (FIG. 13), but appeared at an earlier retention time in HPLC.

A. Partial-Thickness Wounds

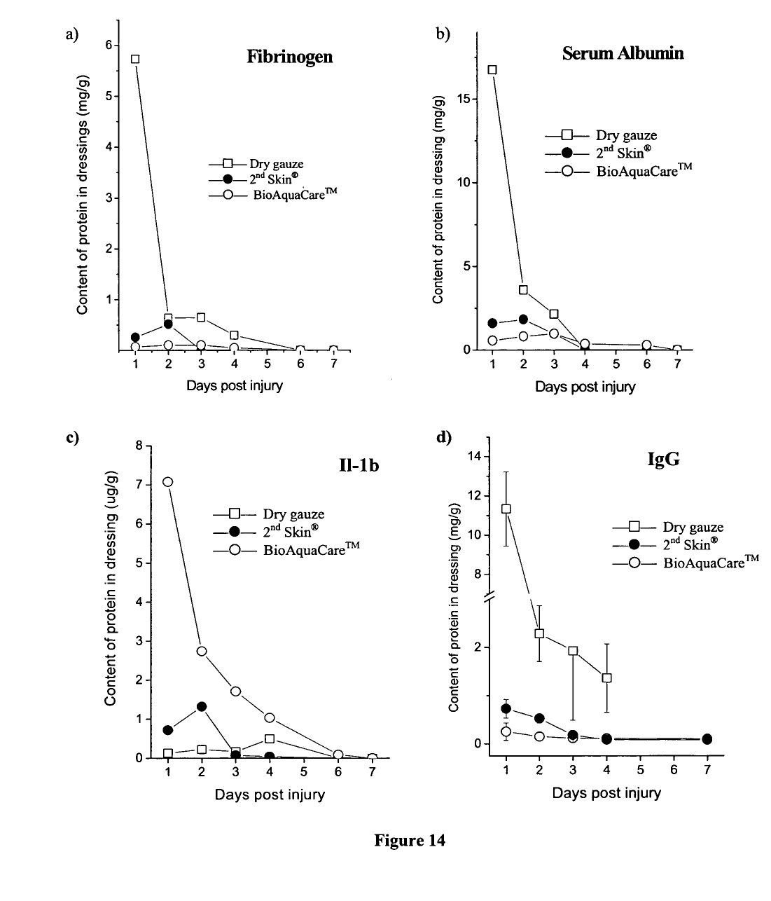

FIGS. 14a-d compare the adsorption profiles of fibrinogen, serum albumin, IL-1β, and IgG by the four dressings over the healing period. It can be seen that regardless of which protein was monitored and which dressing was used, the amount of proteins that were adsorbed decreased over time. However, large variations were observed among the initial amount of proteins that were adsorbed by the different dressings. Specifically, while the adsorption profiles of fibrinogen, serum albumin, and IgG, are somewhat similar, the adsorption profile of IL-1β is sharply different from the other three proteins.

Referring to FIGS. 14a, 14b, and 14d, it was observed that a much larger amount of fibrinogen, serum albumin and IgG were recovered from the dry gauze dressings than from either BioAquaCare™ or 2^{nd} Skin® during what was typically the inflammation phase of the wound healing process (day 0 to day 5). The amount of these three proteins that were extracted from the hydrogel materials was almost 50 times less than the amount that were recovered from the dry gauze dressings. Additionally, it was observed that the amount of fibrinogen, serum albumin and IgG that was adsorbed by 2^{nd} Skin® was consistently larger than the amount that was adsorbed by BioAquaCare™. This is most notable on day 1 and day 2.

Referring to FIG. 14c, an opposite phenomenon was observed for IL-1β. It can be seen from FIG. 14c that among the three dressings studied, the most IL-1β was recovered from BioAquaCare™, the least being from the dry gauze dressings. The difference was about two orders of magnitude. Some adsorption of IL-1β was observed with 2^{nd} Skin®, which was somewhat more than dry gauze on days 1 and 2.

B. Full-Thickness Wounds

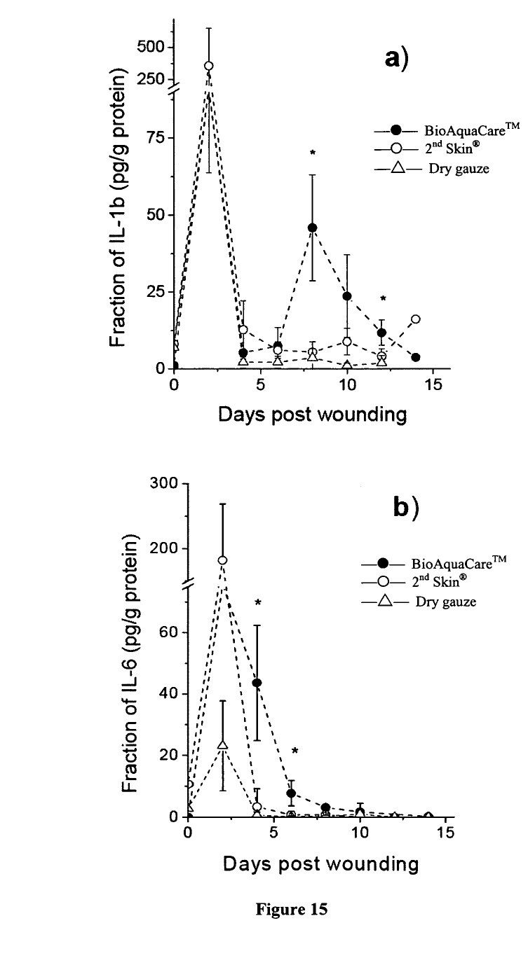

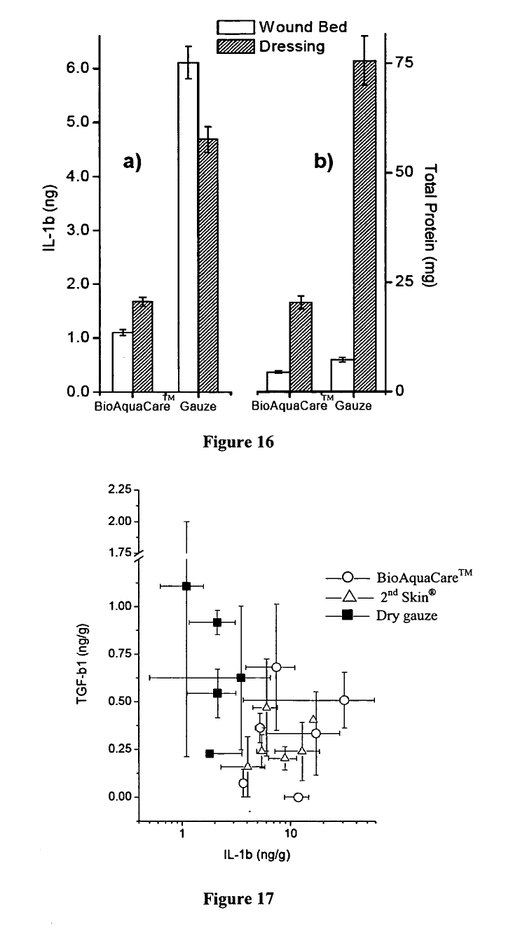

Referring to FIG. 15, a rapid increase in IL-1β content was observed on day 2, irrespective of the wound dressing used (FIG. 15a). This increase is symptomatic of a preliminary inflammatory response to an injury. Starting from day 6, BioAquaCare™ was found to continuously absorb IL-1β from the wound site until the end of the monitoring period, i.e., on day 14. In the period between days 6-14, the fraction of this cytokine extracted from BioAquaCare™ was found to be 2-3 times higher compared to both 2^{nd} Skin® and dry gauze (FIG. 15a). A similar phenomenon was observed when the content of IL-6 was studied (FIG. 15b). In view of the relatively low total protein content found to be absorbed by BioAquaCare™, these data suggested that low-molecular-weight proteins such as IL-1β and IL-6 were selectively absorbed by the hydrophilic matrix of BioAquaCare™. This hypothesis was confirmed when the residual concentration of IL-1β in wound beds was determined and compared with its concentration recovered from the dressings, as shown in FIG. 16. Indeed, BioAquaCare™ appeared to be more absorbent for interleukin IL-1β than dry gauze. As shown in FIG. 16, even though wound fluid absorption by dry gauze resulted in almost complete removal of all available proteins from the wound bed, the concentration of IL-1β in the wound bed remained high.

With regard to TGF-β1, no significant amount was detected in BioAquaCare™ 4 days after wounding, although other types of dressing products contained traceable amounts of TGF-β1. FIG. 17 shows the content of TGF-β1 over time as compared to IL-1β.

Surprisingly, all data points were found to be grouped into two categories symptomatic for either dry or moist wound healing situation. Dry wound healing appears to be characterized by a relatively low level of IL-1β and an elevated secretion of TGF-β1, while wound healing under moist occlusive conditions promotes an inverse relationship between IL-1β and TGF-β1 levels.

Relating the biochemical data obtained in this study to the clinical and histological observations made in Examples 1 and 2, it was concluded that the reduction and moderation of the topical inflammatory response observed with the use of BioAquaCare™ in the treatment of both partial-thickness wounds and full-thickness wounds can be attributed largely to the selective removal of certain cytokines (e.g., IL-1β and IL-6) from the wound site and/or the minimal adsorption of fibrinogen, serum albumin, and IgG to its surface. Adsorption of fibrinogen to the surface of a biomaterial has been shown to induce conformational changes in fibrinogen, which transfers it into proinflammatory state and leads to exposure of binding epitopes for adhesion and accumulation of inflammatory cells. See Hu et al. (2001), BLOOD, 98(4): 1231-1238. Hu et al. showed that structural changes of fibrinogen molecule are mediated by the loss of the hydration shell surrounding the biopolymer. It is now known that adsorbed fibrinogen is pro-inflammatory, while soluble fibrinogen is not. Id. The hydrating properties of BioAquaCare™ therefore also help to reduce inflammatory response by preventing the denaturation of fibrinogen.

Without wishing to be bound by any particular theory, it is believed that the high water content of BioAquaCare™ helps prevent adsorption of certain high-molecular-weight proteins such as fibrinogen, serum albumin, and IgG, to its surface. The water content of hydrated BioAquaCare™ has been shown to be well above 90% by weight. See, e.g., U.S. patent application Ser. No. 10/970,349. Although BioAquaCare™ is a hydrogel matrix largely composed of PEG-protein conjugates, it is believed that the remarkably low content of the polymer component, compared to the extremely high water content, helps preclude the formation of a stable surface for any significant protein adsorption. Without wishing to be bound by any particular theory, it is further believed that the small amount of fibrinogen and IgG that might have been adsorbed to the surface of BioAquaCare did not provide enough ligands for adhesion and activation of neutrophils.

Unlike fibrinogen, serum albumin, and IgG, which are relatively large proteins (fibrinogen, IgG, and serum albumin have a molecular weight of 240 kDa, 140 kDa, and 66 kDa, respectively), IL-1β and IL-6 have a molecular weight of about 17 kDa and about 21.5-28 kDa, respectively, and are therefore much smaller in volume. Without wishing to be bound by any particular theory, it is believed that while 2^{nd} Skin® has a very dense polymeric structure with relatively low porosity, which allows protein adsorption to its surface but no diffusion of proteins across its structure, the composition of the hydrogel matrix found in BioAquaCare™ has sufficiently high porosity to allow absorption of small molecules such as IL-1β and IL-6. It is therefore believed that any absorption of solutes by BioAquaCare™ is selective, being dependent on the molecular size of the absorbed molecules. This selectivity may be due to the unique structural characteristics of the BioAquaCare™ hydrogel matrix formed by PEG and protein molecules, in which the two components form a three-dimensional structure with relatively large pores. Small solutes (e.g. with a molecular size comparable to that of IL-1β or IL-6) may easily penetrate through the hydrogel-tissue interface and then diffuse within the volume of the network by gradient forces. Such diffusion across the polymeric matrix can prevent reactive compounds, such as cytokines including IL-1β and IL-6, from interacting with inflammatory cells on the wound site.

Acute inflammatory response is characterized by a series of local and systemic reaction and accompanied by stimulation of the cells to produce cytokines such as IL-1, IL-6 and TNFα. The cytokines released by macrophages further influence the activity of the surrounded cells. It is becoming commonly accepted that the resident cells of skin are no less important in the generation of a cutaneous inflammatory response. See Kupper (1990), J. CLIN. INVEST., 86: 1783-89. IL-1 is a particularly important molecule in this series of events. Kupper confirmed the inflammatory activity of IL-1 by showing that subcutaneous injections of IL-1 could lead to the generation of a local inflammatory infiltrate. It was also demonstrated that IL-1β can stimulate overproduction of fibrinogen and fibrin, which may interfere with normal wound repair. See Buni et al., “Fibrin/Fibrinogen,” in Encyclopedic Reference of Vascular Biology and Pathology, 107-25 (A. Bikfavli ed., Springer-Verlag 2000).

It thus became evident that the proper management of the level of IL-1 in repairing tissue appears to be of great importance and can represent an instrument for modulating the intensity and duration of the inflammatory phase associated with wound healing. Cessation of the acute inflammatory phase is believed to restore physiological homeostasis, when cellular signaling events are re-directed to stimulation of resident fibroblast and epithelial cells, which initiates the cell proliferation phase of wound healing. The reduction and moderation of acute inflammatory reactions were found to accelerate wound repair as demonstrated in Example 1.

Additionally, it has been observed that until late fetal stages there is generally no sign of connective-tissue scar where the wound has healed. See, Martin, P. (1997), SCIENCE, 276: 75-81 (1997). It has been shown that there is a strong correlation between the age of onset of scarring and the first stage in development when a noticeable inflammatory response is raised after wound. See, e.g., Adzick et al. (1985), J. PEDIATR. SURG., 20: 315-19; Whitby et al. (1991), DEVELOPMENT, 112: 651-68; Hopkinson et al. (1994), J. CELL. SCI., 107(5): 1159-67. By modulating the inflammatory phase of wound healing, it is believed that skin, instead of being repaired, is closer to being regenerated, and accordingly, any scarring that may otherwise occur is minimized if not prevented.

Variations, modifications, and other implementations of what is described herein will occur to those of ordinary skill in the art without departing from the spirit and the essential characteristics of the invention. Accordingly, the scope of the invention is to be defined not by the preceding illustrative description but instead by the following claims, and all changes that come within the meaning and range of equivalency of the claims are intended to be embraced therein.

What is claimed is:

1. A method of moderating and/or shortening a topical inflammatory response, the method comprising:
removing one or more cytokines from a topical site without substantially altering at the topical site a local concentration of one or more plasma proteins.

2. The method of claim 1, wherein the one or more cytokines include at least one of interleukin-1β and interleukin-6.

3. The method of claim 1, wherein the one or more plasma proteins are selected from serum albumin, immunoglobulin G, and fibrinogen.

4. The method of claim 1, comprising hydrating the topical site.

5. The method of claim 1, comprising contacting the topical site with a physiological buffer.

6. The method of claim 1, comprising restoring the local osmolarity of the topical site.

7. The method of claim 1, comprising contacting the topical site with at least one of an antimicrobial or an anticoagulant.

8. The method of claim 1, wherein the topical site comprises an open wound.

9. The method of claim 8, wherein the method accelerates the healing of the open wound.

10. The method of claim 8, wherein the method accelerates wound closure.

11. The method of claim 8, wherein the method prevents a scar at the topical site.

12. The method of claim 1, comprising applying a hydrogel composition to the topical site, the hydrogel composition comprising a protein component and a biocompatible polymer component, wherein the protein component is covalently crosslinked by the biocompatible polymer component.

13. The method of claim 12, wherein the protein component comprises one or more proteins selected from bovine serum albumin, human serum albumin, lactalbumin, ovalbumin, soy albumin, pea albumin, hydrolyzed soy protein, hydrolyzed wheat protein, casein, and combinations thereof.

14. The method of claim 12, wherein the biocompatible polymer component comprises polyethylene glycol or a derivative thereof.

15. The method of claim 12, wherein at least 90% by weight of the hydrogel composition is water.

16. The method of claim 12, wherein the hydrogel composition comprises an antimicrobial.

17. The method of claim 12, wherein the hydrogel composition comprises an anticoagulant.

18. The method of claim 12, wherein a surface of the hydrogel composition is resistant to adsorption of at least one of serum albumin, immunoglobulin G, and fibrinogen.

19. A method of treating a topical inflammatory response, the method comprising applying to a topical site a hydrogel composition comprising a protein component and a biocompatible polymer component, wherein the protein component is covalently crosslinked by the biocompatible polymer component; and removing biological molecules having a molecular weight of less than about 30 kDa.

20. The method of claim 19, wherein the biological molecules include at least one of interleukin-1β and interleukin-6.
